(19) United States
(12) Patent Application Publication
(10) Pub. No.: US 2016/0345918 A1
(43) Pub. Date: Dec. 1, 2016

(54) INTRA-ORAL X-RAY SYSTEM

(71) Applicants: Young Bae Kwon, Seoul (KR); Joong Hyun Choi, Seoul (KR)

(72) Inventors: Young Bae Kwon, Seoul (KR); Joong Hyun Choi, Seoul (KR)

(21) Appl. No.: 14/479,335

(22) Filed: Sep. 7, 2014

(51) Int. Cl. A61B 6/14; A61B 6/00

(52) U.S. Cl. CPC A61B 6/145; A61B 6/542

(57) ABSTRACT

An intra-oral x-ray system combines several tube voltages with varying differences by a pulse method and emits the voltages in one x-ray irradiation to obtain an x-ray imaging with clear contrast. Before x-ray irradiation, the user can preset and save the tube voltage data in the x-ray system by the size of the patient, the area, the purpose of diagnosis and the detector sensitivity. The user can select the saved data or with discretion change the exposure conditions.

### FIELD OF THE INVENTION

The present invention relates to an intra-oral x-ray system for generating several x-ray tube voltages. In detail, it relates to an intra-oral x-ray system for combining several tube voltages by a pulse method and generating the voltages in one x-ray irradiation.

### BACKGROUND OF THE INVENTION

In general, a medical x-ray imaging device provides an appropriate amount of x-rays to a filming area to obtain an image of the desired body part. On the opposite side of the body part, a film or a digital detector is located to transform the penetrating x-ray energy into an imaging. To obtain a desired imaging for a diagnosis, an appropriate output of x-rays should be emitted to the exposure area. X-ray irradiation is determined by tube voltage, tube current and exposure time.

The tube voltage is a potential difference applied between the cathode and the anode of the x-ray tube, and its peak value is usually expressed in Kilovolts (kV). The tube voltage determines x-ray quality, and the load voltage applied to both ends of the x-ray tube in generating x-rays is expressed as the peak value (kV). The tube current is the anode current that flows by electron beams colliding over the anode of the x-ray tube during x-ray irradiation, and its average value is expressed as milli-amperes (mA). The tube current determines the x-ray dose as well as the exposure time. The exposure time indicates the time, usually in seconds (Sec) that the x-ray dose rate has the effective value for irradiation. The exposure time may be displayed separately, but it is usually expressed integrated with the tube current as mAs, the value calculated by multiplying the tube current by the exposure time.

The x-ray exposure dose is proportionate to the square of the tube voltage. When the tube voltage increases, mAs should be reduced to level off the exposure dose on the plane of incidence. Increase in the tube voltage not only leads to increase in the exposure dose, but it also increases the average energy of x-rays, thereby enhancing x-ray quality. When the tube voltage is raised, x-rays better penetrate into the patient and thus, photographic density is thickened even in with the same incident dose. Output exposure dose is proportionate to the tube current and the exposure time. When the exposure time is long, sharpness of the imaging is deteriorated due to movement of body organs or swaying motion of agitated patients. Therefore, the exposure time should be determined by the part of the body or the purpose of photographing.

Bones absorb x-rays better than soft tissues. Strength of penetrating x-rays varies depending on density of the respective human tissues. Except for bones, all other tissues have similar x-ray absorbency and thus, it is not easy to identify an anatomical structure using a general x-ray system.

High density area in the tissue has high x-ray absorbency, resulting in white color, while low density area has high x-ray penetration, appearing in dark color in the imaging. The imaging is displayed by the patient contrast caused by differences in brightness between the two contiguous images, and differences in x-ray attenuation coefficient in several tissues affect the imaging. Strength of x-rays going through the patient is determined by energy and strength of the exposed x-rays, and it vanes depending on absorption of element comprising of the patient, and the degree of scattering (in relation with density and thickness).

Doctors or radiologists, depending on the exposure area and the purpose of diagnosis, calibrate the tube voltage, tube current and exposure time and then apply an x-ray to obtain a desired imaging. Usually, in an intra-oral x-ray system the dentist applies one fixed tube voltage and tube current in the fixed range of 60 kv 70 kV in tube voltage and 1.0 mA-10 mA in tube current, respectively. An x-ray exposure dose is determined by modifying the exposure time. A good-quality radiographic imaging is essential to a dentist in order to focus on examining soft tissues or osseous tissues, Inner structure, location, range and size, boundary and shape of the focus, as well as its relations with the surrounding anatomical structure, is obtained by reading the x-ray imaging.

The transmission factor of the patient is important to examine the focus, and using a proper tube voltage is one of the important elements. If the tube voltage is higher than the appropriate value, x-rays will go through the patient and a dark imaging is generated. In the case of lower tube voltage, x-rays will not go through the patient, resulting in a white imaging, which will lead to poor contrast with contiguous tissues. If the respective tube voltages that fit the transmission factor of several tissues are generated in one x-ray irradiation, the contrast of the desired tissues will be more clearly displayed in the imaging.

To set the tube voltage for the respective tissues, doctors or radiologists can preset and save the desired conditions in the x-ray system before x-ray irradiation.

### SUMMARY OF THE INVENTION

It is an object of this invention to combine several tube voltages with varying potential differences by a pulse method in an intra-oral x-ray system to generate the several tube voltages in one x-ray irradiation. The present inventions provides an x-ray system that allows doctors or radiologists to preset several tube voltages with varying potential differences before an x-ray irradiation and generate the tube voltages thereof in one exposure time. The user of this invention may obtain x-ray imaging with clear contrast of the contiguous tissues in an intra-oral x-ray system that combines several tube voltages with varying potential differences which correspond to different transmission factors of tissues by a pulse method and emits the voltages thereof during one x-ray exposure time.

### BRIEF DESCRIPTION OF THE DRAWINGS

FIG. 1 illustrates a driver and switch circuit diagram of high frequency inverter in accordance with this invention;

FIG. 2 illustrates a circuit diagram of high voltage tube tank in accordance with this invention;

FIG. 3 illustrates a high voltage divide circuit diagram for lowering high voltage to low voltage in accordance with this invention;

FIG. 4 illustrates a general block diagram of the system;

FIG. 5 illustrates an analog circuit diagram for selecting and calibrating the tube voltage;

FIG. 6 illustrates a PWM circuit diagram;

FIG. 7 illustrates a spectrogram of the tube voltages generated in the same percentage terms;

FIG. 8 illustrates a spectrogram of the tube voltages generated in different percentage terms;

FIG. 9 illustrates a tube voltage spectrogram of the existing system that uses one tube voltage;

FIG. 10 illustrate an example of a chart for use of the intra-oral x-ray system of this invention for an upper jaw; and

FIG. 11 illustrate an example of a chart for use of the intra-oral x-ray system of this invention for a lower jaw.

### DETAILED DESCRIPTION OF THE INVENTION

In one embodiment the intra-oral x-ray system of this invention is comprised of an x-ray tube for generating x-rays, a high voltage transformer for generating high voltages, a high voltage rectification circuit for transforming and boosting AC voltage to DC voltage, a high voltage divide circuit for lowering high voltage to low voltage to measure and calibrate high voltages, as well as a high voltage tube tank, a high frequency inverter circuit for generating high voltages, a control printed circuit board for controlling, saving, analyzing and displaying the data and a power printed circuit board for supplying power to the circuit and apparatus in housing.

The intra-oral system of this invention can be embedded in one or several cases, and except for the radiation opening in the x-ray system, all units are completely shielded by lead or high-density materials, protecting patients and users from unnecessary exposure of radiation.

Batteries, or a DC or AC power supply is used to operate the x-ray system, and such a power supply is embedded in the same case as the x-ray system or in a separate housing with a cable connection.

The intra-oral x-ray system of this invention uses a high voltage generator, operated by a pulse width modulation method, such that the tube voltage is modified by the width of an input pulse in the inverter circuit. The user of the system can enter the desired tube voltage data in the numerical value (%) by means of a user interface, such as a keyboard, a mouse or a key switch; when one x-ray irradiation time is set as 100%, the exposure time (%) of the first tube, the exposure time (%) of the second tube, and similarly the exposure time of the tenth tube can be entered in percentage terms.

The tube voltage data entered is routed through the microprocessor to select the respective tube voltages in consecutive order. The selected tube voltage data compares the pulse from the pulse generator that produces the concerned tube voltage, with the voltage from the high voltage tank, provided by dividing the already-generated high voltage, and the pulse in the selected pulse width of high voltage is applied to the inverter circuit input.

The tube voltage with the respective potential differences is combined by the pulse method. The tube voltage is comprised of the first tube voltage, the second tube voltage, and similarly the tenth tube voltage in consecutive order, and a different or the same voltage can be entered for these ten tube voltages. In addition, if no more tube voltages need to be set after a certain number of data, i.e., the first tube voltage, the second tube voltage and the third tube voltage, the preset can be interrupted and only the assigned tube voltage is generated in the order of data preset during the assigned exposure time.

The user can preset the tube voltage and the exposure time in the system based on the size of the patient, the area and sensitivity of the detector; the conditions of x-ray irradiation can be set in advance by category, depending on the size of the patient, the area and sensitivity of the detector, and are saved in the system. The user can select the saved data for future use.

Preferred embodiments of this invention will now be described with reference to the figures. With regard to describing the present invention, it should be noted that the terms used to describe the components of the present invention are intended to correspond to the function performed by the respective components, and they are not intended as a limitation on the technological scope of the present invention.

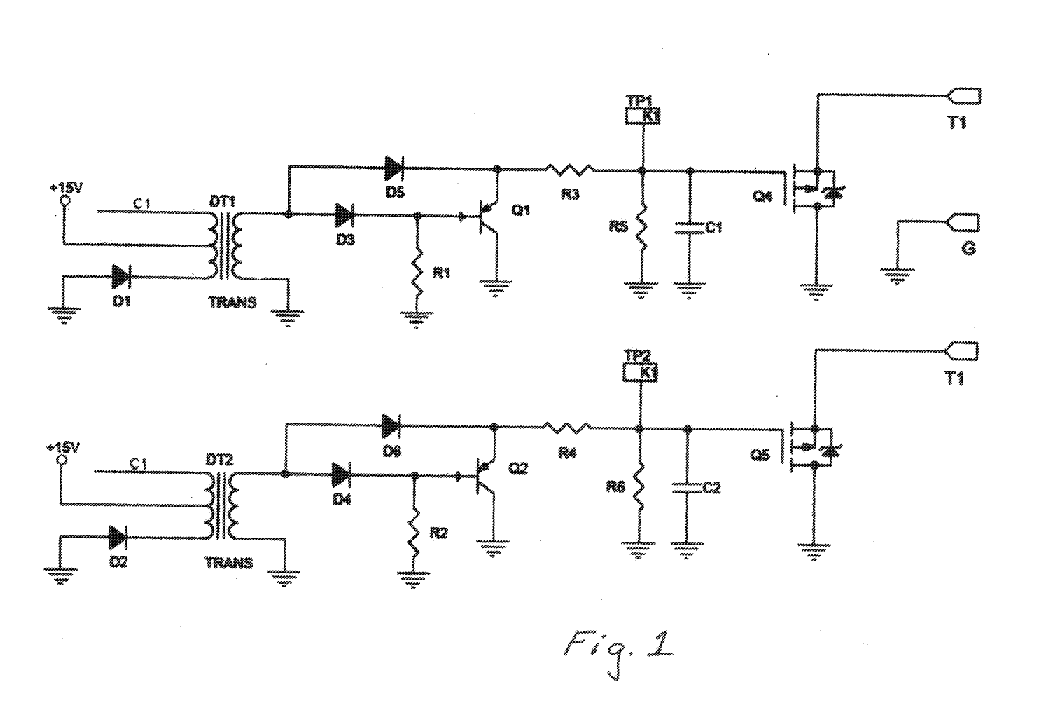

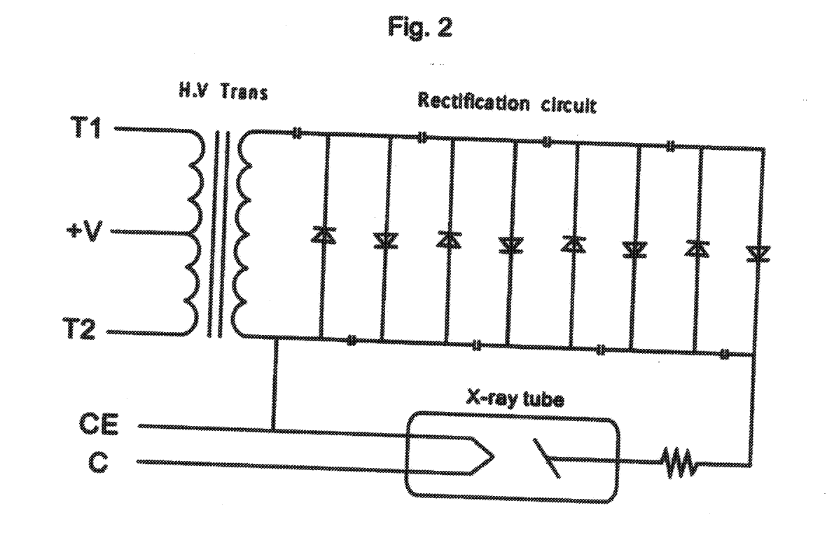

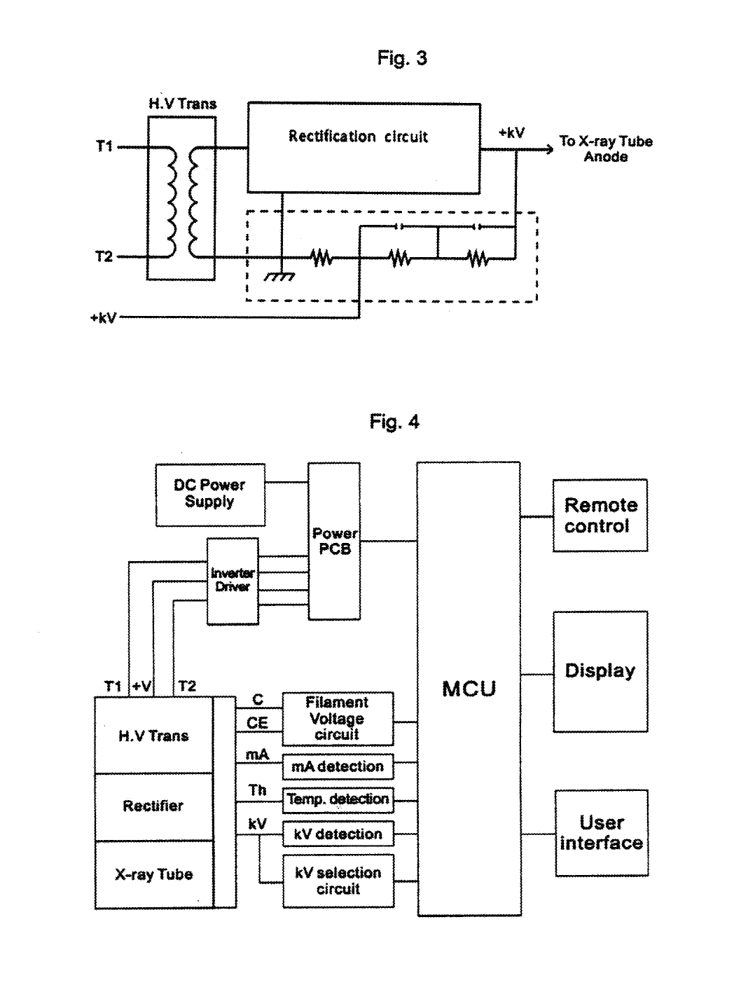

Two high-frequency square pulses from the oscillation circuit are entered into the high frequency inverter circuit as an input signal, respectively, in different time. FIG. 1 illustrates a driver and switch circuit diagram of the high frequency inverter. Output of the inverter circuit is connected to the input circuit (Ti, T2) of the high voltage generator of the high voltage tube tank shown in FIG. 2. The output voltage of the high voltage generator is the input voltage of the high voltage transformer as shown in FIG. 3. The high frequency output voltage of the high voltage transformer is rectified and boosted to DC voltage, which is provided to the anode and the cathode of the x-ray tube for x-ray generation. The output voltage of the high voltage generator has varying potential differences depending on the pulse width received. The high voltage generator includes a dividing circuit to transform high voltage to low voltage measurable in an electronic circuit. In the circuit, high voltage is divided into a constant rate by high resistance and low resistance, which steps down high voltage to low voltage, and the divided voltage is sent to kV control circuit in real-time for monitoring and controlling the high voltage. FIG. 3 illustrates a high voltage divide circuit diagram for lowering high voltage to low voltage.

FIG. 4 illustrates a general block diagram of the system of this invention. MCU is a unit to analyze, save and display the data received from the user and to control the system. The high voltage data that the user sets is saved in MCU, and the respective tube voltage circuits are selected in consecutive order during an x-ray irradiation. The pulse width of the inverter input circuit vanes depending on the tube voltage, and real-time monitoring is available to check if the tube voltage matches with the assigned value.

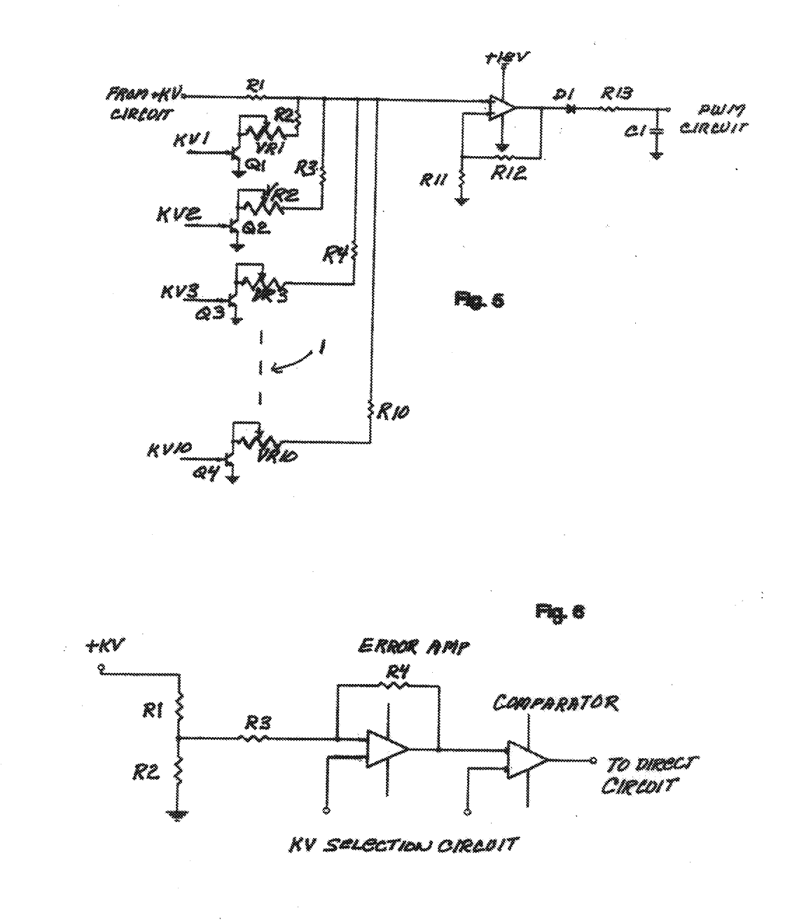

FIG. 5 illustrates an analog circuit diagram for selecting and calibrating the tube voltage. If an error is detected, the pulse width is calibrated by changing the output voltage in the operational amplifier for high voltage controlling. FIG. 6 illustrates a pulse width modulation circuit (PWM) circuit diagram as used in this invention. The pulse width modulation (PWM) control circuit is operated in a method that the output voltage in the high voltage divide circuit is compared with the output voltage in the analog circuit for the respective tube voltages, and the error between the two output voltages is amplified by the error amplifier. The amplified voltage is compared with the saw tooth wave in the comparator and the square wave pulse corresponding to the error is generated. The square wave pulse operates the inverter switch through the drive circuit and stabilizes the high output voltage into the set value. When the output voltage decreases lower than the selected value, the pulse width of square wave can be raised in the same load as the reduced voltage through the error amplifier and the comparator to compensate the decrease in output voltage. On the other hand, if the output voltage increases more than the selected value, the pulse width of square wave can be reduced following the method thereof to return to the setting value.

The tube voltage is selected and exposed in the order of setting. For instance, if the exposure time is set to 1 second, and the first tube voltage is set to 60 kV, the second tube voltage to 62 kV, the third tube voltage to 63 kV, the fourth tube voltage to 65 kV, and the fifth tube voltage to 70 kV, and the respective tube voltages are exposed identically in the same time frame. If the voltages with five varying differences are exposed respectively for 0.2 seconds, the total exposure time of 1 second is completed. Therefore, the setting is made for the first tube voltage to 60 kV (20%), the second tube voltage to 62 kV (20%), the third tube voltage to 63 kV (20%), the fourth tube voltage to 65 kV (20%), and the fifth tube voltage to 70 kV (20%).

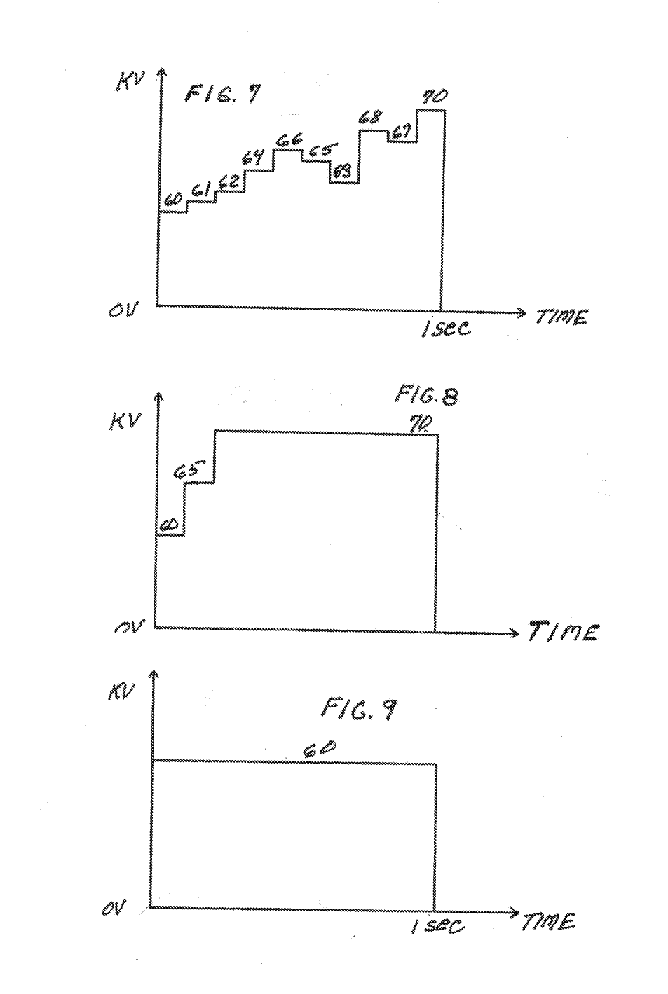

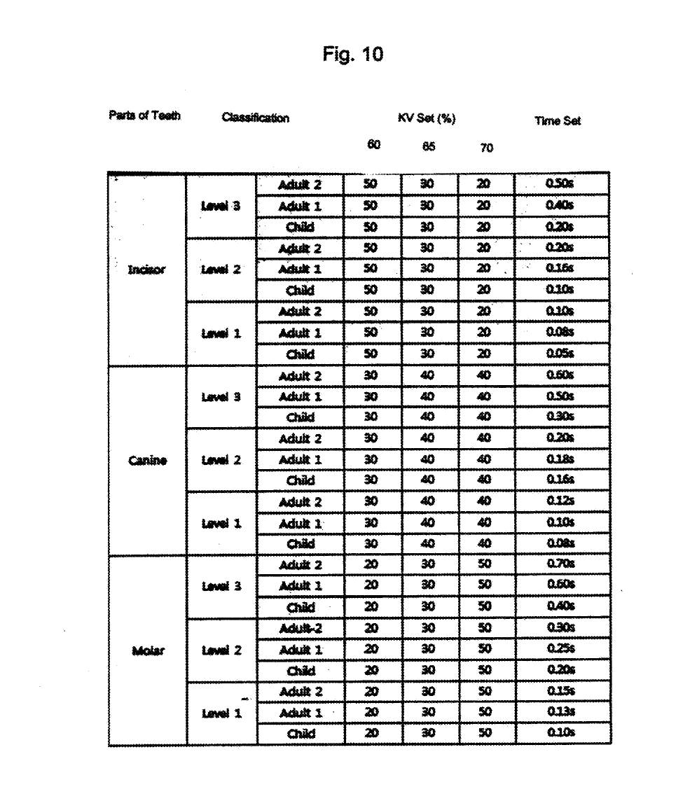

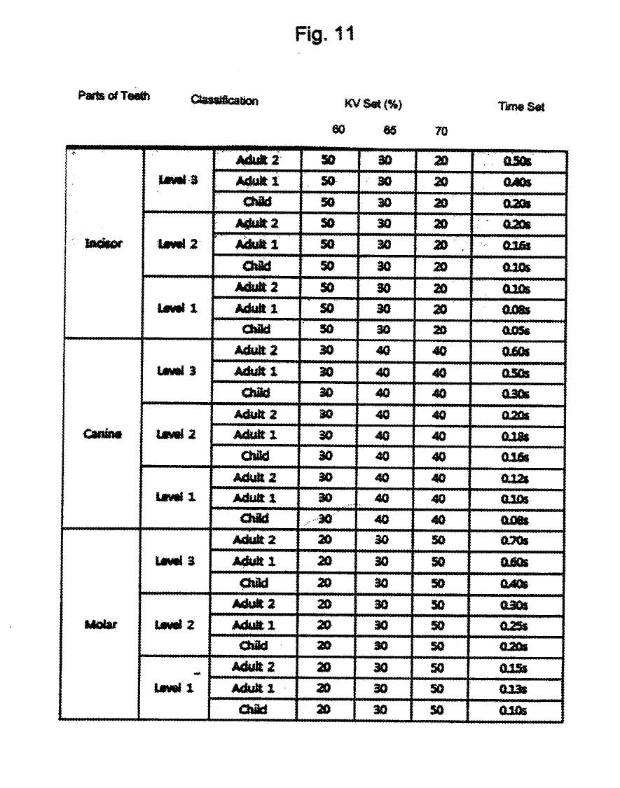

FIG. 7 illustrates a tube voltage spectrogram generated in the same percentage terms. In addition, another setting is available to expose the respective tube voltages in different percentage terms. FIG. 8 illustrates a tube voltage spectrogram generated in different percentage terms. FIG. 9 illustrates a tube voltage spectrogram of the existing system that uses one tube voltage.

A user of the system of this invention can preset the tube voltage and the exposure time in the system by the size of the patient, the area and sensitivity of the detector. For instance, a total of fiftyfour conditions can be preset in a category of six regions of teeth (incisor, canine and molar teeth in the upper and lower jaws), three sizes of the patient (children, adolescents and adults), and three types of detector sensitivity (high, medium and low), and be saved in the system, and the user can select the saved data for use or change the photographing conditions at the users discretion.

Table 1 and Table 2 illustrate examples of charts by the patient and photographing conditions.

Although particular embodiments of the present inventions have been shown and described, it will be understood that it is not intended to limit the present invention to the described embodiments, and it will be obvious to those skilled in the art that various changes and modifications may be made without departing from the spirit and scope of the present invention stated in the scope of claim.

1. An intra-oral x-ray system, wherein several tube voltages with varying differences are combined by a pulse method and emitted in one x-ray irradiation under pre-set conditions; and wherein data preset is available for different tube voltages to be emitted during one exposure time.

2. The intra-oral x-ray system of claim 1, wherein the power is supplied by a battery, or a DC or AC power supply, and the power supply apparatus is embedded in the same case as the x-ray generator or in a separate housing connected by cable to the x-ray generator.

3. The intra-oral x-ray system of claim 1, wherein the tube voltage data is entered or changed by the user.

4. The intra-oral x-ray system of claim 1, wherein the tube voltage is generated consecutively in the setting order during one exposure time.

5. The intra-oral x-ray system of claim 1, wherein one irradiation time is set to 100%, and the exposure time of the several tube voltages are set in the respective percentage terms of the one irradiation time.

6. The intra-oral x-ray system of claim 1, wherein the size of the patient, the purpose of diagnosis and the detector sensitivity, are selected and the tube voltage data is entered based on the selections made.
